(19) United States
(12) Patent Application Publication
(10) Pub. No.: US 2011/0003039 A1
(43) Pub. Date: Jan. 6, 2011

(54) APPARATUS, A CONTAINER AND METHODS THEREOF FOR HEATING FOODSTUFF

(76) Inventor: Yuval Fox, Tel Aviv (IL)

Correspondence Address: Yuval Fox, 255, entrance A, Dizengoff, Tel Aviv, Israel, 63117, omitted

(21) Appl. No.: 12/824,179

(22) Filed: Jun. 27, 2010

Related U.S. Application Data
(60) Provisional application No. 61/213,703, filed on Jul. 6, 2009.

(51) Int. Cl. G01N 33/02; A47J 27/00; B65B 63/08

(52) U.S. Cl. 426/88; 99/331; 53/440

(57) ABSTRACT

In today's lifestyle it is not always practical to be able to prepare foodstuff on a freshly prepared basis. Therefore partial cooking of foodstuff and preparation for heating and completion of cooking is used. An apparatus, a container and methods thereof for heating foodstuff at the home environment provides a solution for preparation of restaurant quality foodstuff. The apparatus, containing water, is adapted to accept the container that contains the foodstuff, where the water temperature is set to a desired temperature that is appropriate for the foodstuff. The container is left within the apparatus for the appropriate amount of time before removal from the apparatus, extracted from the container and served as high quality foodstuff to the table. The container may be used with or without the apparatus and have means to enable handling of the foodstuff in warm to hot water as may be necessary and providing feedback thereof.

This application claims priority from U.S. Provisional Patent Application No. 61/213,703 filed on Jul. 6, 2009, and incorporated herein by reference.

### TECHNICAL FIELD

The present invention relates generally to heating of foodstuff and more specifically to an apparatus, a container and methods thereof for heating foodstuff placed in the container, the container placed in heated water in the apparatus.

### BACKGROUND OF THE INVENTION

The use of frozen products in today's household is common place and provides a quick way of using various products for cooking together with a long period of possible storage under the right conditions. The foodstuff is then typically cooked directly in water or oil, at times after full or partial thawing, baked or grilled, with the food stuff being directly exposed. While known to be a very practical method for fast cooking this cannot be compared to freshly prepared foodstuff. In other cases foodstuff is cooked in a container designed for microwave cooking, however, the quality of the cooking results are debatable at best. Alternatively, the foodstuff, especially if frozen or chilled, is prepared like regular home cooking, however, quality typically falls short from freshly prepared meals. A prior art method suggests the use of porous materials to enable contact between the liquid in which the container is placed. However, while this may be workable for some foodstuff, like the cooking of an omelet from dried eggs, it would be less than desirable when handling, for example a piece of steak, that now would be boiled in a liquid, resulting in undesirable results. In yet another prior art method dry freezing is used before packaging the foodstuff and thereby loosing much of the original consistency of the foodstuff once it is reproduced for eating purposes.

In restaurants, where foodstuff has to be prepared over a relatively short period of time to many guests at various time periods, and where high quality is expected and often demanded, other methods for maintaining freshness of the cooked foodstuff is used. The foodstuff is prepared ahead of time, cocked close to perfection and then chilled. When it is time to be prepared for serving the foodstuff is placed in a container which is then dipped into water at a desired temperature. Cooking is then complete within a few minutes with all the freshness of the product maintained. The large scale systems used for this in restaurants is not a practical solution for household use. Furthermore, it is essential to use the exact temperature of the water and the period of time in which the foodstuff is in contact with the water to maintain the desired result. Heating in water is known in the art to be a superior method to achieve the degree of freshness of such precooked foods.

In view of the limitations of the prior art it would be advantageous to provide an apparatus, a container and methods thereof that overcome these limitations in a manner that would enable the preparation of precooked foodstuff in a restaurant quality at a user's facility.

### SUMMARY OF THE INVENTION

Certain embodiments of the invention include an article for use in the packaging of foodstuff. The article comprises: a container having one or more compartments, each of the one or more compartments designed to contains a foodstuff; a sealing portion for sealing the container for the purpose of preventing air from penetrating said container once air is removed there from; a tear line on the container for opening said container when said foodstuff is ready to be consumed; and an indicator on said container to provide indication of thermal handling of said foodstuff; wherein said article is produced from a non-porous material that conducts heat from an environment in which said container is placed for the purpose of heating said foodstuff to a desired serving temperature, and wherein said non-porous material is further capable of handling a heat range from a low temperature suitable for maintaining the food in storage to a high temperature exceeding the desired heating temperature for final heating of said foodstuff prior to serving.

Certain embodiments of the invention further include an apparatus for heating foodstuff. The apparatus for heating foodstuff comprises: a tub for containing a liquid; means for changing temperature of said liquid in said tub; depressions at the top of said tub designed to accept hinges of at least an article containing foodstuff; and a control unit for receiving information respective of said foodstuff and responsive thereto causing said means for changing temperature to change the temperature of said liquid to a desired temperature; wherein said article comprises: a container having one or more compartments, each of the one or more compartments designed to contains a foodstuff; a sealing portion for sealing the container for the purpose of preventing air from penetrating said container; a tear line on the container for opening said container when said foodstuff is ready to be consumed; and an indicator on said container to provide indication of thermal handling of said foodstuff; wherein said article is produced from a non-porous material that conducts heat from a liquid in which said container is placed for the purpose of heating said foodstuff to a desired serving temperature, and wherein said non-porous material is further capable of handling a heat range from deep freezing to a boiling point of said liquid.

Certain embodiments of the invention further include a method for storing foodstuff in a container. The method for storing foodstuff in a container comprises: cooking the food stuff to a desired degree or preparation; placing the foodstuff in the container; placing an indicator for the container respective of the foodstuff; removing air from the container; sealing said container; and bringing the foodstuff in the container to a desired low temperature level; wherein said container is part of an article comprises: the container having one or more compartments, each of the one or more compartments designed to contains a foodstuff; a sealing portion for sealing the container for the purpose of preventing air from penetrating said container; a tear line on the container for opening said container when said foodstuff is ready to be consumed; and an indicator on said container to provide indication of thermal handling of said foodstuff; wherein said article is produced from a non-porous material that conducts heat from a liquid in which said container is placed for the purpose of heating said foodstuff to a desired serving temperature, and wherein said non-porous material is further capable of handling a heat range from said low temperature level to a high-temperature exceeding the desired heating temperature for final heating of said foodstuff prior to serving.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention will be apparent from the following detailed description taken in conjunction with the accompanying drawings.

FIG. 1 is a schematic diagram side and top views of a foodstuff container in accordance with the invention.

FIG. 2 is a schematic diagram of an apparatus for cooking in accordance with the invention.

FIG. 3 is a schematic diagram of a circuit to operate the apparatus.

FIG. 4 is a flowchart depicting the process of placing foodstuff in a container in accordance with the invention.

### DETAILED DESCRIPTION OF THE INVENTION

The embodiments disclosed by the invention are only examples of the many possible advantageous uses and implementations of the innovative teachings presented herein. In general, statements made in the specification of the present application do not necessarily limit any of the various claimed inventions. Moreover, some statements may apply to some inventive features but not to others. In general, unless otherwise indicated, singular elements may be in plural and vice versa with no loss of generality. In the drawings, like numerals refer to like parts through several views.

In today's lifestyle it is not always practical to be able to prepare foodstuff on a freshly prepared basis. Therefore partial cooking of foodstuff and preparation for heating and completion of cooking is used. An apparatus, a container and methods thereof for heating foodstuff at the home environment provides a solution for preparation of restaurant quality foodstuff. The apparatus, containing water, is adapted to accept the container that contains the foodstuff, where the water temperature is set to a desired temperature that is appropriate for the foodstuff. The container is left within the apparatus for the appropriate amount of time before removal from the apparatus, extracted from the container and served as high quality foodstuff to the table. The container may be used with or without the apparatus and have means to enable handling of the foodstuff in warm to hot water as may be necessary and providing feedback thereof.

Several possible embodiments of the invention shall now be discussed, however, these embodiments are mere examples of the possibilities for manifestation of the disclosed inventions and should not be viewed as a limitation thereon. Furthermore, various combinations of the disclosed invention may be used without departing from the spirit of the invention. Specifically, manual, semi-automatic and fully automatic embodiments of the inventions may be created in accordance with the invention.

Reference is now made to FIG. 1 where a schematic diagram of side and top views of a foodstuff container 100 in accordance with the invention is shown. The materials of the container may differ in various embodiments and include, without limitations, plastics, ceramics, metals and combinations thereof. The heat conduction of such materials must enable the transfer of heat from the water in which the container is to be placed to the foodstuff placed in the container to enable the cooking of the foodstuff to completion. The materials used for the container 100 are non-porous to avoid any direct contact between the foodstuff and the water. The materials may further be capable of being exposed to a range of temperature from the freezing of the foodstuff at temperatures of −25° C. or below, as may be applicable, and up to at least a boiling temperature of water under normal conditions, i.e., 100° C. Other temperature ranges are possible without departing from the scope of the invention and safety margins should always be used.

The container 100 may have hinges 110 used to place the container securely in the apparatus described in more detail herein below. The hinges 110 prevent the container from sinking in the water into which the container is placed for safe removal once cooking is over. In one embodiment of the invention instructions for the cooking in the container 100 are provided in area 120. Area 120 can be a sticker placed on top of the container or, manufactured directly into the container. The instructions would at least provide two instructions: the cooking temperature and the length of time that the container should be placed in the apparatus for the purpose of heating. In another embodiment of the invention area 120 would contain a barcode that has the two cooking parameters, i.e., temperature and time, coded on the bar code. In yet another embodiment of the invention slots may be used at each side of the container, such as slots 140 and 150. The position and number of slots may be used to code the desired temperature and the desired period of time for cooking. For example, a single slot 140 placed towards the upper edge of the container may represent a temperature of 50 degrees Celsius and additional slots may indication additions of several degrees for each such slot. A variety of possibilities for such coding exists and anyone can be used effectively herein. Similarly, the other side of the container, where slots 150 are shown, can provide an indication of time, from, for example 5 minutes all the way to 20 minutes or any other time scale that may be necessary or appropriate for the foodstuff to complete cooking. To enable easy extraction of the foodstuff from the container 100 a tear line 130 is provided. By pulling the top part of the container from, for example, one of the hinges 110, the tear line enables the opening of the container and the extraction of the foodstuff there from.

The container 100 may be used to contain a variety of foodstuff including, without limitations, vegetables, fish and meat in the compartment 160. In one embodiment of the invention the container 100 comprises multiple compartments 160, separated from each other, each such compartment containing a different foodstuff enabling a full meal to be warmed up. For example, such a container may have a first compartment containing a slice of meat, a second compartment containing green beans, and a third compartment containing mashed potatoes. By placing the container in the apparatus, further discussed below, the foodstuff therein is prepared without the need to use three different cooking wares for the process. In a typical embodiment after placing the foodstuff in the container 100, vacuum or near vacuum conditions inside the container 100, prior to sealing the container 100. Alternatively gasses conducive to the long term maintenance of the foodstuff in the container under freezing or chilling conditions may be used, when such gasses replace the oxygen containing air known to expedite the decay of the foodstuff. The container 100 is typically placed in such conditions that cause the foodstuff to freeze or deep-freeze, until such time that the foodstuff is to be cooked, as further explained below. Depending on the foodstuff and specific instructions, the container 100 may be placed to have the foodstuff in a chilling temperature rather than freezing temperature prior to cooking in accordance with the principles of the invention. While the description of the container 100 is with respect to warming the foodstuff contained within the container by placing the container 100 in a liquid, for example, water, for the purpose of heating, it should be noted that other forms of heating suitable for such a container are also possible. For example, the container may be placed in an oven and in this case the atmosphere surrounding the package is the air in the over. In a typical but non-limiting application the air temperature would be between 50 and 100 degrees Celsius.

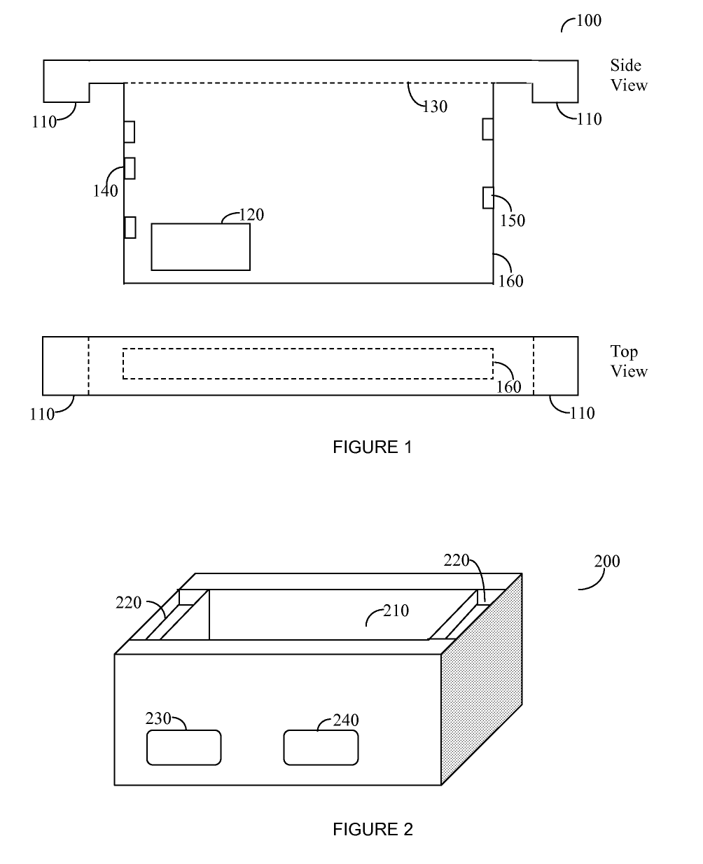

Reference is now made to FIG. 2 where an exemplary and non-limiting schematic diagram of an apparatus 200 for cooking in accordance with the invention is shown. The apparatus is basically a tub 210 that is enabled to contain water and heating the water evenly through the tub 210. On two sides of the apparatus 200 there are depressions 220 that enable the placement of hinges 110 of the container 100 when the container 100 is placed in the tub 210. In one embodiment of the invention sensors are placed in the depression to sense the presence of a container 100 and provide an indication thereof to the control circuit, the control circuit described in more detail below. While the interior of the tub 210 is built from heat conducting materials, the external body of the apparatus 200 uses materials that are either isolated from the internal materials of the tub or otherwise are bad heat conductors so as to maintain the out parts of the apparatus 200 at a temperature that enables handling by a person without particular protection. In one embodiment of the invention the apparatus further includes an input device 230 and a display 240. The input device 230 enables the input of both cooking temperature and cooking time of the foodstuff contained in the container 100. A person using the apparatus 200 with a container 100 would therefore enter the specific cooking temperature and the cooking time for the foodstuff, as shown in area 120, activate the apparatus, and place the container in the tub 210 when instructed to do so. Upon completion of the cooking time display 240 may provide an indication of completion, and in one embodiment, an sound signal may be further or alternatively be generated. In another embodiment of the invention a barcode reader is placed in apparatus 200 and by reading the barcode setting the apparatus 200 automatically to the desired cooking temperature and cooking time. In yet another embodiment the slots on both sides of the container 100, i.e., slots 140 and 150, are detected from within the tub 210 and provided to the control circuit discussed in more detail below. The control circuit then translates the position of the slots to a setting for the cooking temperature and the cooking time.

While the apparatus 200 was described with respect to the insertion of a single container 100 an artisan would readily realize that a plurality of containers can also be placed in such a container. In a multi-container environment the depressions in the apparatus 200 designed to accept the hinges of the container 200 are designed to allow for a separation between containers placed in the tub 210 as it is essential that all surfaces of the container come in contact directly with the heated water. An artisan would appreciate that the more containers to be placed in the apparatus 200 the larger the apparatus, the more water required and more power for heating the water. Therefore, in one embodiment of the invention an accordion style design (not shown) is used for the tub 210. This allows extending of the apparatus 200 to be able to contain additional containers only as necessary and further overcomes the need to heat an unnecessary capacity of water. The apparatus 200 further includes an indication of the maximum amount of water allowed depending on the number of containers placed therein. In one embodiment of the apparatus 200 a spillover passage is provided to enable removal of excess water without flooding the apparatus 200.

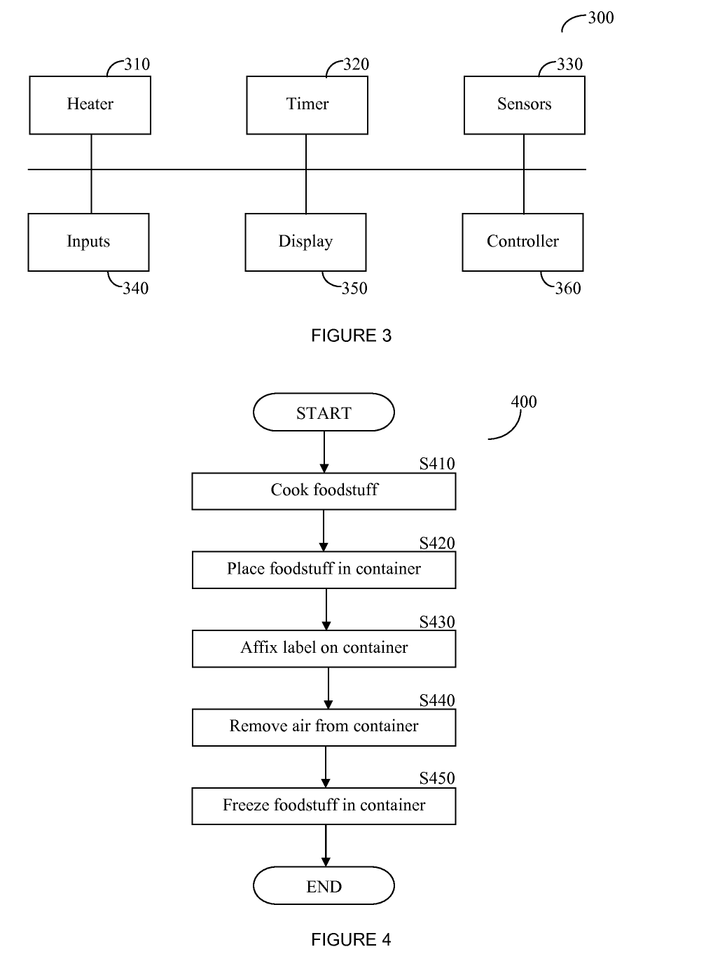

In FIG. 3 there is shown an exemplary and non-limiting diagram of a circuit 300 to operate the apparatus in accordance with the principles of the invention. The circuit contains a heating element 310, a timer 320, sensors 330, inputs 340, a display 350, and a controller 360. The heating element 310 is designed to heat the water contained in tub 210 of apparatus 200 to a desired temperature. In one embodiment heat sensor is one of the sensors 330 and provides feedback to controller 360 respective of the temperature of the water in the tub 210. Accordingly the controller 360 may activate the heating element 310 to maintain the temperature of the water at a desired temperature. Various control schemes may be used, including on-off, proportional, and others well-known in the art. A timer 320 may be activated by controller 360 responsive of a selection of a time period for cooking of the foodstuff in the container 100 placed in the tub 210. Upon completion of the time period the timer 320 signals the controller 360 of the event and controller 360 suspends further heating of the water and provides an indication of a completion of the cooking period. The input 340 may contain a variety of input devices such as, but not limited to, a keyboard, barcode reader, a slot reader, or combination thereof, depending on the specific implementation of the apparatus 200. The display 350 may provide instructions to the user as to next steps to be taken as well as a feedback with respect to entry of various inputs. The sensors 330 may contain a sensor detecting the existence of water in the tub 210 to prevent the case where heating takes place without water being placed in the tub 210. Display 360 may be used to display an error message when insufficient amount of water is in the tub 210. While a display 360 is described, it may also include an audio feedback that provides one or more signals to indicate various steps of the process of heating the foodstuff.

The container 100, while described with hinges 110, may have an embodiment that lacks such hinges. In this case the container is just dropped in the warm or hot water and when ready taken out, torn open, and the foodstuff removed for the purpose of consumption. The container itself maybe heat sensitive changing color as part of the warming process. When the internal temperature of the product reaches the desired heat the container either entirely or partially changes color, either gradually or immediately. The user who placed the container in the water may now reclaim the container and access the foodstuff. In one embodiment the label 120 is made of such color changing material. In yet another embodiment an integrated circuit (IC) is placed in or on the container, for example as part of label 120, the IC having a temperature sensor and radio frequency (RF) transmission capabilities. The IC can continuously measure the temperature of the foodstuff, and cause the transmission of data, for example to a sensor 330 of the circuit 300, providing indication of the temperature of the foodstuff. In another embodiment the IC may further provide indication to the circuit 300 about the content of the foodstuff for the purpose of display on display 350, as well as providing the cooking profile to the circuit 300 of the foodstuff.

Reference is now made to FIG. 4 where an exemplary and non-limiting flowchart 400 depicts the process of placing foodstuff in a container in accordance with the invention. In S410 the foodstuff is cooked to a desired level of perfection. In some embodiments the foodstuff is place in the container without precooking, only after cleaning and peeling, as would be the case for certain vegetables. In S420 the foodstuff is placed in a container such as container 100 described hereinabove in more detail. In S430 the label 120 is placed on the container 100. As noted in the invention, other possible indications other than the placing of the label 120 are possible, in these are specifically included herein, for example, the placement of an IC as part of the container label. In S440 air is removed from the container 100, for achieving vacuum or near vacuum conditions, and then the container 100 is hermetically sealed. In one embodiment gasses that typically do not contain oxygen replace the air in the container, when having such atmosphere is favorable for the maintenance of the freshness of the foodstuff. In S450 the container 100 containing the foodstuff is placed for freezing so that the foodstuff freezes and is available for storage for extended periods of time.

While the cooking period was described herein as a period of constant temperature for a single period of time an artisan would readily realize that other heating profiles are possible. For example, increase over a first period of time of the temperature to a desired temperature, maintaining the temperature at that level for a second period of time, and letting the foodstuff cool down, either naturally or by inducing a cooling period, for a third period of time, and then providing an indication of completion. Other profiles are also possible, may further be coded on the container in similar ways as described hereinabove. Additionally, while foodstuff is specifically described herein, other materials requiring heating that may benefit from the heating in warm or hot water, are specifically included as part of this invention. Foodstuff includes, but is not limited to, processed and unprocessed vegetables, meats, and dairy products. For example, a container 100 may contain mashed potatoes as foodstuff. Alternatively a container 100 may contain a steak cooked to perfection, and to any desired level between rare and well-done, and placed in the container in accordance with the principles f the invention. Then placed in water, in the case of water based heating, or in an oven, both as further discussed hereinabove, and within a predefined time period receive a restaurant quality cooking of the foodstuff. Non-limiting examples, demonstrated by inventor to be suitable for the teachings of the invention, for foodstuff that is pre-cooked and then placed and sealed in accordance with the invention include, but are not limited to: steak, mashed potatoes, salmon in teriyaki sauce, sweet potatoes with garlic and herbs, white fish with herbs and olive oil, chicken breast in date honey, shrimps in chili butter, meat balls in a tomato sauce, risotto, split bean soup, beef Stroganoff, and the likes, and various combinations thereto where each type of foodstuff is placed in a different compartment of the container 100.

Certain principles of the invention may be implemented as hardware, firmware, software or any combination thereof. Moreover, the software is preferably implemented as an application program tangibly embodied on a program storage unit or computer readable medium. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such computer or processor is explicitly shown. In addition, various other peripheral units may be connected to the computer platform such as an additional data storage unit and a printing unit.

All examples and conditional language recited herein are intended for pedagogical purposes to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What we claim is:

1. An article for use in the packaging of foodstuffs comprising:
a container having one or more compartments, each of the one or more compartments designed to contains a foodstuff;
a sealing portion for sealing the container for the purpose of preventing air from penetrating said container once air is removed there from;
a tear line on the container for opening said container when said foodstuff is ready to be consumed; and
an indicator on said container to provide indication of thermal handling of said foodstuff;
wherein said article is produced from a non-porous material that conducts heat from an environment in which said container is placed for the purpose of heating said foodstuff to a desired serving temperature, and wherein said non-porous material is further capable of handling a heat range from a low temperature suitable for maintaining the food in storage to a high temperature exceeding the desired heating temperature for final heating of said foodstuff prior to serving.

2. The article of claim 1, wherein said container is enabled to placed said foodstuff under vacuum.

3. The article of claim 1, wherein said foodstuff is placed in a gaseous environment conducive to maintaining the freshness of said foodstuff.

4. The article of claim 1, wherein said low temperature is one of: deep-freeze temperature and chilling temperature.

5. The article of claim 1, wherein said environment is one of: warm air in an oven or warm liquid in a tub containing said warm liquid.

6. The article of claim 5, wherein said liquid is water.

7. The article of claim 6, wherein said boiling point is at a temperature which is below at least one hundred degrees Celsius.

8. The article of claim 1, further comprising hinges for placing the article in an apparatus for heating said foodstuff.

9. The article of claim 1, wherein said indicator is at least one of:
a label affixed to said container;
an integrated circuit (IC) affixed to said container; and
one or more slots for coding information respective of said foodstuff.

10. The article of claim 9, wherein said label further contains a barcode with indications of heating said foodstuff.

11. The article of claim 9, wherein said IC is enabled for at least one of:
providing information respective of heating of said foodstuff; and
temperature readings of said foodstuff using a temperature sensor integrated into said IC.

12. The article of claim 1, wherein said material is further capable of changing color responsive of heat to provide an indication of heat of said foodstuff.

13. The article of claim 1, wherein said foodstuff is at least one of: steak, mashed potatoes, salmon in teriyaki sauce, sweet potatoes with garlic and herbs, white fish with herbs and olive oil, chicken breast in date honey, shrimps in chili butter, meat balls in a tomato sauce, risotto, split bean soup, and beef Stroganoff.

14. An apparatus for heating foodstuff comprising:
a tub for containing a liquid;
means for changing temperature of said liquid in said tub;
depressions at the top of said tub designed to accept hinges of at least an article containing foodstuff; and
a control unit for receiving information respective of said foodstuff and responsive thereto causing said means for changing temperature to change the temperature of said liquid to a desired temperature;
wherein said article comprises: a container having one or more compartments, each of the one or more compartments designed to contains a foodstuff; a sealing portion for sealing the container for the purpose of preventing air from penetrating said container; a tear line on the container for opening said container when said foodstuff is ready to be consumed; and an indicator on said container to provide indication of thermal handling of said foodstuff; wherein said article is produced from a non-porous material that conducts heat from a liquid in which said container is placed for the purpose of heating said foodstuff to a desired serving temperature, and wherein said non-porous material is further capable of handling a heat range from deep freezing to a boiling point of said liquid.

15. The apparatus of claim 14, wherein said control unit is further capable of at least one of: reading a barcode, reading a slot code, receiving information from an integrated circuit (IC) affixed to said article.

16. The apparatus of claim 14, wherein said control unit is enabled to control said means for changing temperature to a desired temperature profile suitable for said foodstuff.

17. The apparatus of claim 14, wherein said apparatus is extendible to allow placement of additional articles.

18. The apparatus of claim 17, wherein extension of said apparatus is made possible by an accordion design.

19. The apparatus of claim 14, wherein the foodstuff is at least one of: steak, mashed potatoes, salmon in teriyaki sauce, sweet potatoes with garlic and herbs, white fish with herbs and olive oil, chicken breast in date honey, shrimps in chili butter, meat balls in a tomato sauce, risotto, split bean soup, and beef Stroganoff.

20. A method for storing foodstuff in a container comprising:
cooking the food stuff to a desired degree or preparation;
placing the foodstuff in the container;
placing an indicator for the container respective of the foodstuff;
removing air from the container;
sealing said container; and
bringing the foodstuff in the container to a desired low temperature level;
wherein said container is part of an article comprises: the container having one or more compartments, each of the one or more compartments designed to contains a foodstuff; a sealing portion for sealing the container for the purpose of preventing air from penetrating said container; a tear line on the container for opening said container when said foodstuff is ready to be consumed; and an indicator on said container to provide indication of thermal handling of said foodstuff; wherein said article is produced from a non-porous material that conducts heat from a liquid in which said container is placed for the purpose of heating said foodstuff to a desired serving temperature, and wherein said non-porous material is further capable of handling a heat range from said low temperature level to a high-temperature exceeding the desired heating temperature for final heating of said foodstuff prior to serving.

21. The method of claim 20, wherein said low temperature is one of: deep-freeze temperature and chilling temperature.

22. The method of claim 20, wherein said removing air comprises:
bringing said foodstuff to be under vacuum or near vacuum conditions.

23. The method of claim 20, further comprising one of:
adding one or more gasses conducive for maintaining the foodstuff's freshness; and
replacing said air by one or more gasses conducive for maintaining the foodstuff's freshness.

24. The method of claim 20, wherein said indicator is at least one of:
a label affixed to said container;
an integrated circuit (IC) affixed to said container; and
one or more slots for coding information respective of said foodstuff.

25. The method of claim 24, wherein said label further contains a barcode with indications of heating said foodstuff.

26. The method of claim 24, wherein said IC is enabled for at least one of:
providing information respective of heating of said foodstuff; and
temperature readings of said foodstuff using a temperature sensor integrated into said IC.

27. The method of claim 20, wherein the foodstuff is at least one of: steak, mashed potatoes, salmon in teriyaki sauce, sweet potatoes with garlic and herbs, white fish with herbs and olive oil, chicken breast in date honey, shrimps in chili butter, meat balls in a tomato sauce, risotto, split bean soup, and beef Stroganoff.
